(19) United States
(12) Patent Application Publication
(10) Pub. No.: US 2010/0183687 A1
(43) Pub. Date: Jul. 22, 2010

(54) PROCESS FOR PREPARING PARTICLES OF OPIOIDS AND COMPOSITIONS PRODUCED THEREBY

(76) Inventors: D. Phillip Cox, Audubon, PA (US); James D. Talton, Gainesville, FL (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/692,170

(22) Filed: Jan. 22, 2010

Related U.S. Application Data
(60) Provisional application No. 61/146,358, filed on Jan. 22, 2009.

(51) Int. Cl. A61K 31/485; A61K 9/14

(52) U.S. Cl. 424/400; 514/282; 977/773

(57) ABSTRACT

The present invention provides compositions comprising particles comprising a poorly soluble opioid drug and a stabilizer, wherein the particles have an average diameter of less than about 10,000 nm. Methods of making the compositions and their use as pharmaceutical compositions for treating disorders such as pain are also described.

This application claims priority from U.S. Provisional Application Ser. No. 61/146,358 filed Jan. 22, 2009, the contents of which is hereby incorporated by reference in its entirety.

### FIELD OF THE INVENTION

The present invention relates generally to compositions comprising particles of opiate drugs and to pharmaceutical compositions comprising such compositions. It further relates to methods of making such particle compositions, and to methods of making the corresponding pharmaceutical compositions. The particles comprise an opiate drug or a mixture of an opiate drug and a stabilizer milled to an effective particle size of less than 10,000 nm. The particle compositions described herein allow poorly soluble opiate drugs to be administered more effectively by routes such as oral, sublingual, buccal, parenteral, intravenous, inhalation or transdermal administration.

### BACKGROUND

Oral administration of drugs is generally preferred for reasons of patient comfort and compliance. However, many drugs, including many opioids, are poorly soluble at neutral pH, and are thus are poorly or variably absorbed when delivered orally. This may also lead to delayed onset-of-action, which may be undesirable if being used for the treatment of pain. Consequently, many such drugs are administered through more invasive routes, such as by inhalation, sublingual, buccal, subcutaneous, transdermal or intravenous routes, which result in more rapid onset of action and/or more complete absorption.

Delivery of therapeutic agents to the respiratory tract is important for both local and systemic treatment of disease. Attempts to develop respirable aqueous suspensions of poorly soluble compounds have been unsuccessful. Micronized therapeutic agents suspended in aqueous media are too large to be delivered by aerosolized droplets. Large particles (greater than 10 μpm) are primarily deposited on the back of the throat. Greater than 60% of the particles with sizes between 1 and 10 μm pass with the air stream into the upper bronchial region of the lung where most are deposited. With particles less than 1 μm, essentially all of the particles enter the lungs and pass into the peripheral alveolar region; however, about 70% are exhaled and therefore lost.

Injectable nanoparticles may sometimes be a more desirable route of administration than oral administration for certain active drugs. Nanoparticulate formulations afford the possibility to prepare parenteral formulations of water-insoluble or poorly water-soluble drugs. These formulations are by their nature suspensions rather than solutions since their particles are dispersed/suspended in a pharmaceutically acceptable vehicle. Liposomes, emulsions and colloids, when used as carriers for an active drug, are also suspensions rather than solutions.

For example, injectable formulations of naproxen are preferable over oral administration forms for several reasons. First, such formulations can lessen or eliminate side effects of gastro-intestinal irritation. Second, intravenous (IV) or intra-muscular (IM) administration of a drug results in a significantly shorter response time as compared to oral administration. Moreover, injectable formulations of pain medication are also preferable for post-operative health care, where oral administration may not be feasible. Injectable naproxen formulations are difficult to formulate due to the low solubility of naproxen. Moreover, the soluble formulations of injectable naproxen are undesirable because they produce intense pain and/or a burning sensation upon administration. Lee and De Castro (U.S. Pat. No. 6,153,225) describe the methods for making and using an injectable formulation of nanoparticulate naproxen that produces minimal or no pain or burning sensation upon administration.

Several approaches for improving the oral delivery of poorly soluble drugs are well-known in the art. For example, poorly soluble drugs may be in the form of more dissolvable salts (oxycodone HCl), administered as dispersions in large amounts of fatty acids, or milled to yield nanoparticles. There has been substantial effort in the last decade to produce drug particles from 100 nanometers to a few microns because of their improved dissolution properties (especially with insoluble drugs) and ability to be absorbed more efficiently. However, each of those approaches suffers from certain drawbacks, such as, e.g., inadequate stability, difficulty of manufacture, adverse interactions with the drug to be delivered, or the use of toxic amounts of adjuvants or inhibitors.

Dispersible nanoparticulate compositions, as described in U.S. Pat. No. 5,145,684 (“the 684 patent”), are particles less than approximately 400 nanometers in size consisting of a poorly soluble therapeutic or diagnostic agent having absorbed onto or associated with the surface thereof a non-crosslinked surface stabilizer. The '684 patent does not describe nanoparticulate compositions of opiate derived analgesics or antagonists. Methods of making nanoparticulate compositions are also described, for example, in U.S. Pat. Nos. 5,518,187 and 5,862,999, both for “Method of Grinding Pharmaceutical Substances”, and U.S. Pat. No. 5,510,118 for “Process of preparing therapeutic compositions containing nanoparticles”. Nanoparticles are prepared by dispersing a drug substance and surface modifiers in water and wet grinding in the presence of rigid grinding media, such as silica beads or a polymeric resin. These methods require removal of the grinding media and drying as additional steps to generate a dry nanoparticles product.

Cryogenic jet-milling with nitrogen is a well-suited size reduction technique for pharmaceutical powders that may be chemically degraded by mixing in aqueous media. Using cryogenic conditions while milling easily oxidized or heat-sensitive materials controls chemical decomposition, which can protect and enhance final product properties, produce finer particles/improve nanoparticle size yield, and increase the production rate (does not require additional steps for wet media milling described above). One method for cryogenic jet-milling with nitrogen is described in US patent application 20080029625.

Recent ICH guidelines require that oxycodone hydrochloride compositions contain reduced amounts of 14-hydroxycodeinone relative to current commercially available oxycodone hydrochloride. 14-Hydroxycodeinone belongs to a class of compounds designated as potential gene-toxins due to their susceptibility to the Michael addition reaction. 14-hydroxycodeinone may also be formed during the conversion of oxycodone base to oxycodone hydrochloride due to the conversion of 7,8-dihydro-8,14-dihydroxycodeinone (DHDHC) to 14-hydroxycodeinone by dehydration (see US patent application 20080132703). Thus, methods of preparation of oxycodone, as well as dry particle size reduction techniques that reduce the levels of the 14-hydroxycodeinone, are necessary.

### SUMMARY

The invention provides a composition comprising particles comprising a poorly soluble opioid drug and a stabilizer, wherein the particles have an average diameter of less than about 10,000 nanometers (nm).

In some embodiments, the invention provides a composition comprising particles of a poorly soluble drug (such as, e.g., an opioid). For example, in some embodiments, the invention provides a composition comprising particles of oxycodone encapsulated by poly-vinyl-pyrollidone (PVP), wherein the oxycodone content ranges from about 10% to about 90%.

In other embodiments, the invention provides pharmaceutical compositions comprising such encapsulated compositions. Such pharmaceutical compositions may, in some embodiments, further comprise at least one excipient. In other embodiments, such pharmaceutical compositions may further comprise a second compound such as, e.g., a second drug, including, e.g., an opioid receptor antagonist, an anti-inflammatory drug, or an analgesic. For example, in some embodiments, the invention provides a pharmaceutical composition comprising a composition comprising the PVP-encapsulated oxycodone composition and a pharmaceutically acceptable carrier.

In some embodiments, the invention provides a first method of making a composition comprising particles of a poorly soluble drug, the method comprising:
blending a poorly soluble drug together with a stabilizer to form a mixture;
processing said mixture to form coarse particles having an average diameter ranging from about 0.1 mm to about 5 mm; and
processing said coarse particles to form fine particles having an average diameter ranging from about 100 nanometers to about 10,000 nm.

In yet other embodiments, the invention provides a method of treating pain, comprising administering a therapeutically effective amount of the pharmaceutical composition described above to a patient in need thereof.

### DETAILED DESCRIPTION

### I. Particulate Delivery Systems

The invention provides a composition comprising particles comprising a poorly soluble opioid drug and a stabilizer, wherein the particles have an average diameter of less than about 10,000 nm.

In an embodiment, the particles have an average diameter of less than about 1000 nm. In another embodiment, the particles have an average diameter of less than about 550 nm.

In another embodiment, the poorly-soluble opioid drug is not an opioid receptor antagonist. In another embodiment, the poorly-soluble opioid drug is not methyl naltrexone or a pharmaceutically acceptable salt thereof (e.g., methyl naltrexone bromide). In an embodiment, the poorly-soluble opioid drug is not a mixed opioid receptor agonist/antagonist (e.g., buprenorphine, nalbuphine).

In still another embodiment, particles comprise a poorly soluble opioid drug encapsulated by a stabilizer. In other embodiments, the poorly soluble opioid drug is oxycodone and the stabilizer is poly-vinyl-pyrollidone.

In some embodiments, the invention provides a composition (also referred to as a particulate delivery system or PDS) comprising particles of a poorly soluble drug encapsulated by a stabilizer. In some embodiments, those particles are fine particles, and have a diameter of less than 3 mm, less than 2 mm, less than 600 μm, less than 500 μmm, or less than 300 μm. In some embodiments, the fine particles have an average diameter ranging from about 0.1 mm (100 μm) to about 3 mm. For example, the particles may have a diameter of less than 2.06 mm (corresponding to a 10 mesh sieve), less than 1.68 mm (corresponding to a 12 mesh sieve), less than 1.40 mm (corresponding to a 14 mesh sieve), less than 1.20 mm (corresponding to a 16 mesh sieve), less than 1.00 mm (corresponding to an 18 mesh sieve), less than 0.853 mm (corresponding to a 20 mesh sieve), less than 0.710 mm (corresponding to a 25 mesh sieve), less than 0.599 mm (corresponding to a 30 mesh sieve), or less than 0.500 mm (corresponding to a 35 mesh sieve). In other embodiments, the particles may have a diameter of less than 300 μm, and may be able to pass through a 50 mesh sieve.

As used herein, the term drug encompasses the corresponding free base or hydrate, salt, prodrug, solvate (including a mixed solvate), or complex (such as a pharmaceutically acceptable complex, and/or a complex with a polymer).

As used herein, the terms poorly soluble drug, drug having poor solubility, and the like refer to a drug (in its neutral (i.e., uncharged) state) having a water solubility at neutral pH of less than 10 mg/ml. In some embodiments, the drug (in its neutral state) has a water solubility at neutral pH of less than 5 mg/ml. In other embodiments, the drug (in its neutral state) has a water solubility at neutral pH of less than 1 mg/ml. For example, oxycodone base (i.e., uncharged oxycodone) has a solubility at neutral pH of <1 mg/ml (whereas the corresponding hydrochloride salt has a solubility at neutral pH of 100 mg/ml). Thus, as used herein, oxycodone (including oxycodone base and its salts, hydrates, solvates, complexes, etc.) is a poorly soluble drug. Similarly, morphine base (i.e., uncharged morphine) has a solubility at neutral pH of <1 mg/ml (whereas the corresponding sulfate has a solubility at neutral pH of 64 mg/ml). Thus, as used herein, morphine (including morphine base and its salts, hydrates, solvates, complexes, etc.) is a poorly soluble drug.

In certain embodiments, the poorly soluble drug is chosen from opioids (including opiates). Opioids include naturally-occurring, synthetic, and semi-synthetic opioids, including, but not limited to, alfentanil, allylprodine, alphaprodine, anileridine, benzylmorphine, bezitramide, buprenorphine, butorphanol, clonidine, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazine, fentanyl, heroin, hydrocodone, hydromorphone, hydroxypethidine, isomethadone, ketobemidone, levallorphan, levomethadyl, levorphanol, levophenacylmorphan, lofentanil, meperidine, meptazinol, metazocine, methadone, methylnaltrexone bromide, metopon, morphine, myrophine, nalbuphine, naloxone, naltrexone, narceine, nicomorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propiram, propoxyphene, remifentanil, sulfentanil, tramadol, and tilidine. For example, in some embodiments, the opioid may be chosen from, e.g., buprenorphine, codeine, fentanyl, hydrocodone, hydromorphone, morphine, methylnaltrexone, nalbuphine, nalmefene, oxymorphone, oxycodone, pethidine, and tramadol.

Thus, a poorly soluble opioid drug refers to an opiod drug (in its neutral (i.e., uncharged) state) having a water solubility at neutral pH of less than 10 mg/ml. In some embodiments, the opioid drug (in its neutral state) has a water solubility at neutral pH of less than 5 mg/ml. In other embodiments, the opioid drug (in its neutral state) has a water solubility at neutral pH of less than 1 mg/ml. For example, oxycodone base (i.e., uncharged oxycodone) has a solubility at neutral pH of <1 mg/ml (whereas the corresponding hydrochloride salt has a solubility at neutral pH of 100 mg/ml). Thus, as used herein, oxycodone (including oxycodone base and its salts, hydrates, solvates, complexes, etc.) is a poorly soluble opioid drug. Similarly, morphine base (i.e., uncharged morphine) has a solubility at neutral pH of <1 mg/ml (whereas the corresponding sulfate has a solubility at neutral pH of 64 mg/ml). Thus, as used herein, morphine (including morphine base and its salts, hydrates, solvates, complexes, etc.) is a poorly soluble opioid drug.

In an embodiment, the poorly-soluble opioid drug is an opioid receptor agonist. In an embodiment, the poorly soluble opioid drug is selected from alfentanil, allylprodine, alphaprodine, anileridine, benzylmorphine, bezitramide, buprenorphine, butorphanol, clonidine, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazine, fentanyl, heroin, hydrocodone, hydromorphone, hydroxypethidine, isomethadone, ketobemidone, levallorphan, levomethadyl, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propiram, propoxyphene, remifentanil, sulfentanil, tramadol, and tilidine, or a pharmaceutically acceptable salt thereof.

In another embodiment, the poorly soluble opioid drug is selected from alfentanil, allylprodine, alphaprodine, anileridine, benzylmorphine, bezitramide, butorphanol, clonidine, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazine, fentanyl, heroin, hydrocodone, hydromorphone, hydroxypethidine, isomethadone, ketobemidone, levallorphan, levomethadyl, levorphanol, levophenacylmorphan, lofentanil, meperidine, meptazinol, metazocine, methadone, metopon, morphine, myrophine, narceine, nicomorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propiram, propoxyphene, remifentanil, sulfentanil, tramadol, and tilidine, or a pharmaceutically acceptable salt thereof.

In another embodiment, the poorly soluble opioid drug is oxycodone or a pharmaceutically acceptable salt thereof.

In some embodiments, the PDS may further comprise an additional compound, such as an additional drug. The additional drug may be chosen from, e.g., opioid receptor antagonists (including μ-receptor antagonists), opioid receptor agonists (including μ-receptor agonists), mixed μ-agonists/μ-antagonists, anti-inflammatory drugs, and analgesics. In some embodiments, the second drug is an opioid receptor antagonist, such as, e.g., the μ-opioid receptor antagonist naloxone, including naloxone.HCl (naloxone hydrochloride). In some embodiments where the poorly soluble drug is an opioid analgesic, the opioid receptor antagonist is added to deter abuse of the opioid analgesic.

The poorly soluble drug may be present in an amount ranging from about <1% to about 100% of the PDS by mass. For example, the poorly soluble drug may be present in an amount ranging from about 0.01% to about 90%, about 0.01% to about 10%, about 0.2 to about 5%, about <1% to about 10%, about 0.01% to about 10%, about 0.1% to about 10%, about 0.01% to about 5%, about 0.1% to about 5%, about 0.1% to about 3%, about <1% to about 50%, about <1% to about 30%, about <1% to about 80%, about 5% to about 90%, about 10% to about 90%, about 10% to about 95%, or about 0.1 to about 5% of the PDS, by mass. In some embodiments, the poorly soluble drug content may be about 0.5% by mass.

As used herein, the term “stabilizer” refers to a compound other than a pharmaceutically active agent used in the PDS for the purpose of inhibiting growth or preventing re-aggregation of the active agent particle.

In some embodiments, the stabilizer is a polymer, such as, e.g., a water-soluble polymer, a polymer of neutral charge, or a water-soluble polymer of neutral charge. In some embodiments, the stabilizer is biodegradable. In some embodiments, the stabilizer is bioerodable. In some embodiments, the stabilizer may be considered by the FDA to be generally regarded as safe (GRAS).

In some embodiments, the stabilizer is a polymer chosen from polyethylene oxide (also known as polyethylene glycol or PEG), polypropylene oxide, or copolymers thereof. In some embodiments, the stabilizer is a water-soluble polymer of neutral charge chosen from polyethylene glycol, polyvinyl alcohol, polyvinylpyrrolidone (PVP), block copolymers of ethylene oxide and propylene oxide such as, e.g., poloxamers, and tetrafunctional block copolymers derived from sequential addition of propylene oxide and ethylene oxide to ethylenediamine.

In some embodiments, the stabilizer may have an average molecular weight of about, e.g., 500, 1000, 2000, 3000, 3350, 3500, 4000, 4500, 5000, 6000, 8000, 10,000, or 100,000 Daltons (Da), or an average molecular weight ranging from, e.g., about 100 Da to about 100,000 Da, about 100 Da to about 6,000 Da, about 500 Da to about 5000 Da, about 1000 Da to about 4000 Da, about 2000 Da to about 4000 Da, about 2000 Da to about 6000 Da, about 1000 Da to about 10,000 Da, or about 3000 Da to about 4000 Da.

For example, the stabilizer may be a PVP, such as a PVP of average molecular weight of about, e.g., 17,000 (K17) or 30,000 (K-30) Daltons.

The stabilizer may be present in an amount ranging from about <1% to about 100% of the PDS by mass. For example, the stabilizer may be present in an amount ranging from about 0.01% to about 90%, about 0.01% to about 10%, about 0.2 to about 5%, about <1% to about 10%, about 0.01% to about 10%, about 0.1% to about 10%, about 0.01% to about 5%, about 0.1% to about 5%, about 0.1% to about 3%, about <1% to about 50%, about <1% to about 30%, about <1% to about 80%, about 5% to about 90%, about 10% to about 90%, about 10% to about 95%, or about 0.1 to about 5% of the PDS, by mass.

### II. Methods of Making PDS

In some embodiments, the invention provides a first method of making a composition (such as those described in Section I) comprising particles of a poorly soluble drug encapsulated by a stabilizer, the method comprising:
blending a poorly soluble drug together with a stabilizer to form a mixture;
processing (e.g., by mixing or granulating) said mixture to form coarse particles having an average diameter ranging from about 0.1 mm to about 5 mm; and
processing (e.g., by jet-milling) said coarse particles to form fine particles having an average diameter ranging from about 100 nm to about 10,000 nm.

In an embodiment, the fine particles are milled to an average diameter of less than about 1000 nm. In another embodiment, the fine particles are milled to an average diameter of less than about 550 nm. In another embodiment, the fine particles are milled using cryogenic jet-milling.

Another embodiment is the method wherein the fine particles comprise the poorly soluble opioid drug encapsulated by the stabilizer. In other embodiments, the poorly soluble opioid drug is oxycodone and the stabilizer is poly-vinyl-pyrollidone.

In certain embodiments, the fine particles have an average diameter ranging from about 0.1 μm to about 3 mm. Particulate materials, also designated as “particles”, to be produced in accordance with this invention are those in which small nanometer to micrometer size particles are desirable. Examples might include nanoparticles and microparticle forms of pharmaceuticals, including poorly soluble drugs. The possibilities and combinations are numerous.

In general, the setup includes a venturi-type nozzle or ‘Tee’ valve to introduce cryogenic gas to a jet mill. Combinations of dry gases at cryogenic temperatures (generally below 0° C.) before introduction into the jet mill is used to eliminate moisture-induced agglomeration, as well as promote brittle fracture of particles upon impaction, and has been observed to act synergistically to produce a marked improvement in the particle size reduction efficiency. Cryogenic liquids suitable for use in this method include liquid argon, liquid nitrogen, liquid helium or any other liquified gas having a temperature sufficiently low to produce brittle fracture of particles. The cryogenic liquid also prevents milling losses and thermal damage to the feed material that would otherwise be caused by the volatization or overheating of constituent ingredients. A powder is placed in a temperature controlled vessel, such as a jacketed hopper or a screw-feeder, or is frozen beforehand. The cryogenic liquid and gas inputs are opened and the flow and temperature is set to the desired process conditions. The cryogenic gas input system, for example liquid nitrogen mixed with nitrogen gas, may be connected to a standard commercial jet mill, such as a Trost Gem-T (Garlock, Inc., Newton, Pa.), Trost T-15 (Garlock, Inc., Newton, Pa.), Fluid Air Aljet (Fluid Energy Processing and Equipment Co., Telford, Pa.), Hosikawa Alpine AS Spiral Jet Mill (Hosikawa Micron, Ltd., Runcom, Cheshire, UK), Sturtevant Micronizer (SturtevantInc., Hanover, Mass.), or similar system as the main carrier gas in a variety of gas input setups. Pre-run setup of the system may include attaching a temperature probe or flowmeter, such as a TSI Model 4040 Flowmeter or similar system, at the gas input or to the top of the cyclone (in place of air relief bag), setting the carrier gas on different input pressures and documenting the gas flow and temperature measurements (CFM). The milling process may be started by turning on the powder feeder and after passing powder through the milling region, the jet-milled powder is collected in the cup or similar receiver unit (typically particles ˜1-10 microns) or from the bag above the cyclone (particles <1 micron), depending on the exact run conditions. Ideally, to obtain particles less than 1-10 microns, powder from the cup is run through the jet-mill under similar run conditions multiple times, or passes, to obtain a high yield of the desired particle size. Materials suitable for use in this method can include any materials, including peptides, polypeptides, proteins, polymers, small molecule drugs and non-pharmaceutical materials.

In certain embodiments, the fine particles have an average diameter ranging from about 0.1 mm (100 μm) to about 3 mm.

### III. Pharmaceutical Compositions (Final Dosage Forms)

In some embodiments, the invention provides pharmaceutical compositions (sometimes referred to as final dosage forms or FDF) comprising the compositions described in Section I above with one or more pharmaceutically acceptable excipients or carriers.

The poorly soluble drug may be present in the pharmaceutical composition in an amount ranging from about <1% to about 100% by mass. For example, the poorly soluble drug may be present in an amount ranging from about 0.01% to about 90%, about 0.01% to about 10%, about 0.2 to about 5%, about <1% to about 10%, about 0.01% to about 10%, about 0.1% to about 10%, about 0.01% to about 5%, about 0.1% to about 5%, about 0.1% to about 3%, about <1% to about 50%, about <1% to about 30%, about <1% to about 80%, about 5% to about 90%, about 10% to about 95%, or about 0.1 to about 5% of the pharmaceutical composition by mass. In some embodiments, the poorly soluble drug content may be about 0.5% by mass.

In some embodiments, the pharmaceutical compositions further comprise a second compound, such as a second drug. The second drug may be chosen from, e.g., opioid receptor antagonists (including μ-receptor antagonists), opioid receptor agonists (including μ-receptor agonists), mixed μ-agonists/μ-antagonists, anti-inflammatory drugs, and analgesics. In some embodiments, the second drug is an opioid receptor antagonist, such as, e.g., the μ-opioid receptor antagonist naloxone, including naloxone.HCl (naloxone hydrochloride). In some embodiments where the poorly soluble drug is an opioid analgesic, the opioid receptor antagonist is added to deter abuse of the opioid analgesic. The resulting compositions may have reduced potential for abuse of the opioid, relative to compositions that do not comprise an opioid receptor antagonist.

In some embodiments, the pharmaceutical compositions further comprise at least one excipient (such as, e.g., a water-soluble polymer, surfactant, disintegrant and/or enhancer), such as a pharmaceutically acceptable excipient (also referred to herein as a carrier or pharmaceutically acceptable carrier). Examples of pharmaceutically acceptable excipients are described in Remington's Pharmaceutical Sciences by E. W. Martin, and include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. In some embodiments, the pharmaceutical compositions also contain pH buffering reagents, and wetting or emulsifying agents.

The pharmaceutical compositions may, in some embodiments, be formulated for oral administration, for example as tablets, capsules, or other oral dosage forms. Such oral dosage forms may be prepared by conventional means. The pharmaceutical composition can also be prepared as a liquid, for example as a syrup or a suspension. The liquid can include suspending agents (e.g., sorbitol syrup, cellulose derivatives or hydrogenated edible fats), emulsifying agents (lecithin or acacia), non-aqueous vehicles (e.g., almond oil, oily esters, ethyl alcohol, or fractionated vegetable oils), and preservatives (e.g., methyl or propyl-p-hydroxybenzoates or sorbic acid). The preparations can also include flavoring, coloring and sweetening agents. Alternatively, the composition can be presented as a dry product for constitution with water or another suitable vehicle.

For buccal and sublingual administration, the composition may take the form of tablets or lozenges according to conventional protocols.

For administration by oral or nasal inhalation, the compounds for use according to the present invention are conveniently delivered in the form of an aerosol spray from a pressurized pack or nebulizer (e.g., in phosphate buffered saline (PBS)), with a suitable propellant, e.g., dichlorodifluoromethane, trichlorofluoromethane, dichlorotetrafluoromethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The pharmaceutical composition can also be formulated for parenteral administration (including, e.g., intravenous or intramuscular administration) by bolus injection. Formulations for injection can be presented in unit dosage form, e.g., in ampoules or in multidose containers with an added preservative. The compositions can take such forms as suspensions, solutions, or emulsions in oily or aqueous vehicles, and contain formulatory agents such as suspending, stabilizing and/or dispersing agents. Alternatively, the active ingredient can be in powder form for constitution with a suitable vehicle, such as, e.g., pyrogen free water.

The pharmaceutical composition can also be formulated for rectal administration as a suppository or retention enema, e.g., containing conventional suppository bases such as PEG, cocoa butter or other glycerides.

In some embodiments, the pharmaceutical compositions described herein provide improved dissolution of the poorly soluble drug, relative to the unencapsulated poorly soluble drug, and/or to another dosage form (such as, e.g., injectable dosage form). For example, dissolution may be increased by, e.g., at least 10%, 15%, 20%, 25%, 30%, 35%, 40%, 50%, 60%, 70%, 80%, 90%, 93%, 95%, 96%, 97%, 98%, 99%, 100%, 110%, 120%, 130%, 140%, 150%, or 200%, or by, e.g., at least 2, 3, 4, 5, 6, 7, 8, 9, 10, 20, 30, 40, 50, 100, or 1000 fold, as measured by a Vankel tablet dissolution apparatus approved by the United States Pharmacopeia.

In some embodiments, the pharmaceutical compositions described herein provide improved oral bioavailability of the poorly soluble drug, relative to the unencapsulated poorly soluble drug, and/or to another dosage form (such as, e.g., injectable dosage form). For example, absorption may be increased by, e.g., at least 10%, 15%, 20%, 25%, 30%, 35%, 40%, 50%, 60%, 70%, 80%, 90%, 93%, 95%, 96%, 97%, 98%, 99%, 100%, 110%, 120%, 130%, 140%, 150%, or 200%, or by, e.g., at least 2, 3, 4, 5, 6, 7, 8, 9, 10, 20, 30, 40, 50, 100, or 1000 fold, as measured by, e.g., in vivo pharmacokinetic studies in a preclinical animal model or human clinical evaluation.

In some embodiments, the pharmaceutical compositions described herein are immediate-release formulations. In such embodiments, the pharmaceutical compositions provide a more rapid onset of action of the poorly soluble drug, relative to the unencapsulated poorly soluble drug, and/or to another dosage form (such as, e.g., injectable dosage form). For example, the onset of action may be shortened by, e.g., at least 10%, 15%, 20%, 25%, 30%, 35%, 40%, 50%, 60%, 70%, 80%, 90%, 93%, 95%, 96%, 97%, 98%, 99%, 100%, 110%, 120%, 130%, 140%, 150%, or 200%, or by, e.g., at least 2, 3, 4, 5, 6, 7, 8, 9, 10, 20, 30, 40, 50, 100, or 1000 fold, as measured by, e.g., in vivo pharmacokinetic studies in a preclinical animal model or human clinical evaluation.

In other embodiments, the pharmaceutical compositions described herein are controlled-release formulations. In such embodiments, the pharmaceutical compositions described herein provide a more controlled or sustained onset of action of the poorly soluble drug.

In some embodiments, the pharmaceutical compositions described herein have reduced absorption variability, relative to the unencapsulated poorly soluble drug, and/or to another dosage form (such as, e.g., injectable dosage form).

In some embodiments, the pharmaceutical compositions described herein are associated with improved patient compliance, relative to another pharmaceutical composition comprising the same poorly soluble drug (which may be in another dosage form, such as, e.g., injectable dosage form).

### IV. Methods of Making Pharmaceutical Compositions

In further embodiments, the invention provides a method of making a pharmaceutical composition, comprising the first, second, or third method described in Section II above, and further comprising formulating the fine particles.

In certain embodiments, the fine particles are formulated into unit doses.

In embodiments in which the pharmaceutical compositions comprise at least one excipient, the invention also provides a method of making a pharmaceutical composition, comprising the first, second, or third method described in Section II above, and further comprising: (a) mixing the fine particles with at least one excipient to form a second mixture; and (b) formulating the second mixture.

In certain embodiments, the fine particles are formulated into unit doses.

To prepare a pharmaceutical compositions, the particles of poorly soluble opioid drug and stabilizer may be mixed with a pharmaceutical carrier and/or excipient according to conventional pharmaceutical compounding techniques, which carrier may take a wide variety of forms depending of the form of preparation desired for administration (e.g. oral or parenteral). Suitable pharmaceutically acceptable carriers and/or excipients are well known in the art. Descriptions of some of these pharmaceutically acceptable carriers and/or excipients may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

### V. Methods of Treatment

The pharmaceutical compositions described herein are useful to treat any disease or condition for which administration of a corresponding hydrophobic drug is desirable. For example, compositions comprising opioid agonists are useful for the treatment of pain. The terms “treat,” treatment,” and “treating” refer to (1) a reduction in severity or duration of a disease or condition, (2) the amelioration of one or more symptoms associated with a disease or condition without necessarily curing the disease or condition, or (3) the prevention of a disease or condition. Suitable subjects include, e.g., humans and other mammals, such as, e.g., mice, rats, dogs, and non-human primates.

In certain embodiments, the invention provides a method of treating pain, comprising administering a therapeutically effective amount of the pharmaceutical composition described in Section III to a subject in need thereof. For example, in such embodiments, the poorly soluble drug may be chosen from, e.g., opioids, including, e.g., buprenorphine, codeine, fentanyl, hydrocodone, hydromorphone, morphine, methylnaltrexone, nalbuphine, nalmefene, oxymorphone, oxycodone, pethidine, and tramadol. Moreover, in such embodiments, the pharmaceutical composition may further comprise one or more additional active ingredients in addition to the poorly soluble opioid drug, for example an opioid receptor antagonist to deter abuse of the opioid analgesic (e.g., naloxone or naltrexone), an anti-inflammatory drug or an analgesic.

The term “therapeutically effective amount” means that amount of pharmaceutical agent that elicits the biological or medicinal response in a tissue system, animal or human, that is being sought by a researcher, veterinarian, medical doctor, or other clinician, which includes alleviation of the symptoms of the syndrome, disorder or disease being treated.

The term “subject” as used herein, refers to an animal, preferably, a mammal, most preferably, a human, who has been the object of treatment, observation or experiment.

The pharmaceutical compositions may be administered by any means that accomplish their intended purpose. Examples include administration by oral, parenteral, subcutaneous, intravenous, intramuscular, intraperitoneal, transdermal, buccal or ocular routes.

Optimal dosages to be administered may be readily determined by those skilled in the art, and will vary with the particular drug used, the mode of administration, the strength of the preparation, the mode of administration, and the advancement of the condition being treated. In addition, factors associated with the particular patient being treated, including patient age, weight, diet and time of administration, will result in the need to adjust dosages.

The following examples are intended to be purely exemplary of the present invention.

### EXAMPLES

### Example 1

### Preparation of Oxycodone PDS

Oxycodone base and PVP K-30 (ISP) powders (100 g, 80:20 ratio by mass) were mixed in a turbula mixer (Glen Mills) at room temperature for 10 minutes. The mixture was transferred to a glovebox and placed in a Bransonic spoon feeder above a Fluid Air Aljet jet mill. A liquid and gas nitrogen mixture was adjusted resulting in a pressure of 120 psi (+/−10 psi) in each jet. The powder was fed into the mill over approximately 15 minutes and the resulting powder in the cup below the cyclone passed again through the mill five additional passes. The resulting white powder in the bag was obtained with a yield of >80 g, containing particles with a diameter less than 10 microns and highly electrostatic. The particles obtained could be used to make tablets or capsules for oral administration, as well as an injectable suspension or an inhaled dry-powder for faster onset of action.

### Example 2

### Preparation of Immediate-Release Oral Oxycodone Capsules

An immediate-release oral dosage form (gelatin capsules) containing the oxycodone•base/PVP particles prepared in Example 1 was prepared as follows. The PDS prepared in Example 1 was dry mixed with additional PEG 3350 for bulking to achieve the correct capsule fill weight (400-500 mg) to achieve the desired dose. Clear gelatin #1 capsules were then filled with the mixture in a Fast-CAP Filling machine to yield capsules containing 20.0±2 mg oxycodone.

### Example 3

### Dissolution of Immediate-Release Oral Oxycodone Base Capsules

The in vitro dissolution rate of the formulation prepared in Example 2 was measured by USP Paddle Method 2 at 50 or 100 rpm in 1000 ml of deionized water at 37° C. It was found that greater then 75% (by weight) of the oxycodone•base/PVP particles prepared in Example 1 was dissolved after 45 minutes compared to unmilled oxycodone base capsules which dissolved approximately 20% at 45 minutes.

### Example 4

### Preparation of Controlled-Release Pharmaceutical Compositions Comprising Oxycodone

A controlled-release pharmaceutical composition comprising oxycodone is prepared according to the methods of the invention. The compositions may have the following characteristics:

| | |
|---|---|
| Oxycodone content | 0.1-200 mg |
| PVP K-30 content | 100-1000 mg |

A biphasic oral delivery system may be prepared by mixing oxycodone•base/PVP particles prepared in Example 1 (supplying initial effect up to 2 hours) with unmilled oxycodone (sustained 2-8 hour effect).

### Example 5

### Single-Dose Pharmacokinetic Study in Dogs

The milled oxycodone base composition prepared in Example 2 was administered in a capsule via oral gavage to male beagle dogs at a 0.5 mg/kg dose compared to a commercial 5 mg oxycodone HCl capsule (n=3 each). Plasma samples were collected up to 24 hours, and following extraction, oxycodone and oxymorphone (active metabolite of oxycodone) were analyzed using LC-MS-MS. Plasma concentrations observed at the first 15 minute collection time were 1.5 to 2 times higher for the milled oxycodone base formulations compared to commercial oxycodone HCl capsule. This example demonstrated the potential of the described milled oxycodone base formulation for faster onset-of-action for breakthrough pain.

### Example 6

### Preparation of Methylnaltrexone Bromide PDS

Methylnaltrexone bromide powder (100 g) was transferred to a glovebox and placed in a Bransonic spoon feeder above a Fluid Air Aljet jet mill. A liquid and gas nitrogen mixture was adjusted testing various pressures and powder was fed into the mill over approximately 5 minutes. The resulting powder in the cup below the cyclone passed again through the mill several additional passes. The resulting white powder in the bag contained particles with a diameter less than 10 microns and highly electrostatic. The milled Methylnaltrexone bromide and PEG 3,350 (Dow) powder (100 g, 2:98 ratio by mass) was mixed in a turbula mixer (Glen Mills) at room temperature for 10 minutes. Similar to Example 5, the milled methylnaltrexone bromide/PEG 3,350 composition was administered in a capsule via oral gavage to male beagle dogs at a 0.3 mg/kg dose compared to intravenous controls (n=3 each) and plasma samples were taken to measure systemic absorption. The described formulation may be used to make tablets or capsules for oral administration.

While various embodiments of the present invention have been described above, it should be understood that they have been presented by way of example only, and not limitation. The present invention is limited to the preparation of drug nanoparticles of poorly soluble drugs chosen from opioids (including opiates) by a jet mill. The present invention is not limited by the particular design of the jet mill, scale or batch size or by any of the exemplary embodiments, but should be defined only in accordance with the following claims and their equivalents.

It will be understood by those skilled in the art that changes may be made to the above-described embodiments of the invention without departing from the broad inventive concepts thereof. It is understood, therefore, that this invention is not limited to the particular embodiments disclosed, but it is intended to cover all modifications that are within the scope and spirit of the invention as defined by the appended claims.

What is claimed is:

1. A composition comprising particles comprising a poorly soluble opioid drug and a stabilizer, wherein the particles have an average diameter of less than about 10,000 nm.

2. The composition of claim 1, wherein the poorly soluble opioid drug includes naturally-occurring, synthetic, and semi-synthetic opioids.

3. The composition of claim 2, wherein the poorly soluble opioid drug is selected from the group consisting of alfentanil, allylprodine, alphaprodine, anileridine, benzylmorphine, bezitramide, buprenorphine, butorphanol, clonidine, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazine, fentanyl, heroin, hydrocodone, hydromorphone, hydroxypethidine, isomethadone, ketobemidone, levallorphan, levomethadyl, levorphanol, levophenacylmorphan, lofentanil, meperidine, meptazinol, metazocine, methadone, methylnaltrexone bromide, metopon, morphine, myrophine, nalbuphine, naloxone, naltrexone, narceine, nicomorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propiram, propoxyphene, remifentanil, sulfentanil, tramadol, and tilidine, or pharmaceutically acceptable salts thereof.

4. The composition of claim 3, wherein the poorly soluble opioid drug is oxycodone or a pharmaceutically acceptable salt thereof.

5. The composition of claim 1, wherein the particles comprise a poorly soluble opioid drug encapsulated by the stabilizer.

6. The composition of claim 5, wherein the stabilizer is poly-vinyl-pyrollidone.

7. The composition of claim 1, wherein the particles have an average diameter of less than about 1000 nm.

8. The composition of claim 1, wherein the particles have an average diameter of less than about 550 nm.

9. The composition of claim 4, wherein the oxycodone content ranges from about 10% to about 90%.

10. The composition of claim 1, wherein said composition further comprise at least one excipient.

11. A method of making the composition of claim 1, the method comprising:
blending a poorly soluble opioid drug together with a stabilizer to form a mixture;
processing said mixture to form coarse particles having an average diameter ranging from about 0.1 mm to about 5 mm; and processing said coarse particles to form fine particles having an average diameter ranging from about 100 nanometers to about 10,000 nanometers.

12. The method of claim 11, wherein the mixture of the poorly soluble opioid drug and the stabilizer are processed by mixing or granulating to form the coarse particles.

13. The method of claim 12, wherein the coarse particles are processed by jet-milling to form the fine particles.

14. The method of claim 11, wherein the fine particles are milled to an average diameter of less than about 1000 nm.

15. The method of claim 11, wherein the fine particles are milled to an average diameter of less than about 550 nm.

16. The method of claim 11, wherein the fine particles comprise the poorly soluble opioid drug encapsulated by the stabilizer.

17. The method of claim 13, wherein the poorly soluble opioid drug is selected from the group consisting of alfentanil, allylprodine, alphaprodine, anileridine, benzylmorphine, bezitramide, buprenorphine, butorphanol, clonidine, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazine, fentanyl, heroin, hydrocodone, hydromorphone, hydroxypethidine, isomethadone, ketobemidone, levallorphan, levomethadyl, levorphanol, levophenacylmorphan, lofentanil, meperidine, meptazinol, metazocine, methadone, methylnaltrexone bromide, metopon, morphine, myrophine, nalbuphine, naloxone, naltrexone, narceine, nicomorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propiram, propoxyphene, remifentanil, sulfentanil, tramadol, and tilidine, or pharmaceutically acceptable salts thereof.

18. The method of claim 17, wherein the poorly soluble opioid drug is oxycodone or pharmaceutically acceptable salts thereof.

19. The method of claim 13, wherein the stabilizer is poly-vinyl-pyrollidone.

20. A method of treating pain comprising administering to a subject in need thereof a therapeutically effective amount of the composition of claim 1.

21. A pharmaceutical composition comprising the composition of claim 1 and a pharmaceutically acceptable carrier.
